(19) United States
(12) Patent Application Publication
(10) Pub. No.: US 2009/0204056 A1
(43) Pub. Date: Aug. 13, 2009

(54) CONSTANT CURRENT TREATMENT DEVICE

(76) Inventor: Zvi Nitzan, Zofit (IL)

Correspondence Address: KENYON & KENYON LLP, 1500 K STREET N.W., SUITE 700, WASHINGTON, DC, 20005, US

(21) Appl. No.: 12/300,961

(22) PCT Filed: May 17, 2007
(86) PCT No.: PCT/IL07/00602

§ 371 Date: Apr. 27, 2009

Related U.S. Application Data
(60) Provisional application No. 60/800,835, filed on May 17, 2006.

(51) Int. Cl. A61N 1/30; A61N 1/36

(52) U.S. Cl. 604/20; 607/51

(57) ABSTRACT

The present invention provides a device for treatment of a nail condition comprising, at least one active electrode disposed on the nail, at least one counter electrode, at least one power source for providing power to the device, wherein the at least one power source is electrically coupled to the at least one active electrode and the at least one counter electrode; a controllable voltage converter coupled to the at least one power source; and a sensing means coupled to the nail and to the controllable voltage converter, wherein the sensing means is configured to facilitate adjustment of the output voltage of the power source according to the nail resistance to facilitate providing a constant current to the nail with high efficiency. The present invention is also directed to a method of providing a constant current to treat a nail condition.

### FIELD OF THE INVENTION

The present invention relates to a device and method for facilitating constant current. Moreover, the present invention is of a device and method for facilitating constant current in treatment of a nail condition using iontophoresis.

### BACKGROUND OF THE INVENTION

Onychomycosis is a disease of the nail caused by yeast, dermatophytes, or other molds, and represents approximately 50% of all nail disorders. Toenail infection accounts for approximately 80% of onychomycosis incidence, while fingernails are affected in about 20% of the cases. Many methods are known for the treatment of fungal infections, including the oral and topical use of antibiotics (e.g., nystatin and amphotericin B), imidazole antifungal agents such as miconazole, clotrimazole, fluconazole, econazole and sulconazole, and non-imidazole fungal agents such as the allylamine derivatives terbinafine and naftifine, and the benzylamine butenafine.

However, onychomycosis has proven to be resistant to most treatments. Nail fungal infections reside in an area difficult to access by conventional topical treatment and antifungal drugs cannot readily penetrate the nail plate to reach the infection sites under the nail. Therefore, onychomycosis has traditionally been treated by oral administration of antifungal drugs; however, clearly this is undesirable due to the potential for side effects of such drugs, in particular those caused by the more potent antifungal drugs such as itraconazole and ketoconazole. An alternative method of treatment of onychomycosis is by removal of the nail before treating with a topically active antifungal agent; such a method of treatment is equally undesirable. Systemic antimycotic agents require prolonged use and have the potential for significant side effects. Topical agents have usually been of little benefit, primarily because of poor penetration of the antifungal agents into and through the nail mass.

Iontophoresis has been known for many years, as a means to deliver drugs and cosmetic active agents into the skin for therapeutic purposes. While widely used in dermal delivery of active agents, iontophoresis has not been utilized hitherto in the treatment of nail infections. Furthermore, the literature is devoid of pragmatic ways to enable a system, which is practically and conveniently usable for long periods of daily treatment.

In the context of the present invention, the term “iontophoresis” will stand for any method of electrical delivery of substances, such as for example dermal delivery, including electrotransportation, iontophoresis, electroosmosis, electroporation, and/or a combination thereof. The terms “device,” “iontophoretic device,” “iontophoretic patch,” “electrically operated device,” and “electrically operated patch,” as used herein, will interchangeably stand for any method or device, used for electrical delivery of substances, including electrotransportation, iontophoresis, electroosmosis, and electroporation.

It is desirable in treatment of a nail infection such as onychomycosis by iontophoresis to deliver the same dose of active drug to each patient. The concentration of drug delivered is proportional to the current applied and therefore to facilitate delivery of a fixed drug concentration, a constant current is needed. The resistance of the nail to be treated can vary according to many factors, such as age, sex, areas of infection, past or present physical trauma to the nail, smoking, etc.

In individuals with dry and or thick skin on the digit, such as is common in elderly people the nail and digit resistance may be relatively high, necessitating relatively high voltage in order to generate the current required to deliver a medication by iontophoresis. Further, each treatment of onychomycosis may be for substantial lengths of time, such as several hours to several days. As such, the power source should be utilized as efficiently as possible.

One method for facilitating constant current may utilize a fixed voltage constant current source. Such a current source needs to have sufficient voltage to generate the required current into a nail with the highest expected resistance. The power efficiency of such a current source is directly proportional to the nail resistance. As such, the lower the nail resistance is, the lower the efficiency of the current source will be. A nail treatment device using such a method does not take into account the varying resistance of a nail. This may be problematic, when the power source is intended to work for an extended period of time, such as in the treatment of onychomycosis.

It would be advantageous to have an iontophoretic device and method of treating onychomycosis using a constant current source with variable/adaptable output voltage, and whereby the adaptable voltage constant current source can maintain high efficiency for a wide range of nail resistance. In addition, it would be advantageous to have a device for treatment of onychomycosis wherein the output voltage is controlled according to the nail resistance of the patient. It would further be desirable to have an iontophoretic device and method of use for treating onychomycosis, which minimizes the drain of the device power source.

### BRIEF DESCRIPTION OF THE DRAWING

The various features of the invention will best be appreciated by simultaneous reference to the description which follows and the accompanying drawing and in which:

FIG. 1 illustrates a schematic representation of a constant current device according to an embodiment of the present invention.

### DESCRIPTION OF THE INVENTION

Embodiments of the present invention are useful for topically treating onychomycosis, i.e., a disease (e.g., a fungal infection) of the nail plate on the hands or feet. As used herein, reference to a “nail” includes reference to one, or some, or all parts of the nail, including the nail plate (the stratum corneum unguis, which is the horny compact outer layer of the nail, i.e., visible part of the nail), the nail bed (the modified area of the epidermis beneath the nail plate, over which the nail plate slides as it grows), the cuticle (the tissue that overlaps the nail plate and rims the base of the nail), the nail folds (the skin folds that frame and support the nail on three sides), the lunula (the whitish half-moon at the base of the nail), the matrix (the hidden part of the nail under the cuticle), and the hyponychium (the thickened epidermis underneath the free distal end of a nail). Nails grow from the matrix. Nails are composed largely of keratin, a hardened protein (that is also in skin and hair). As new cells grow in the matrix, the older cells are pushed out, compacted and take on the familiar flattened, hardened form of a fingernail or toenail.

Embodiments of the present invention are also useful in treating any other suitable condition of the nail, surrounding area of the nail and/or skin, such as but not limited to viral and/or bacterial infections of the nail and/or skin.

Embodiments of the invention may have several aspects. One aspect is an electrically operated device including a constant current, intended for application on the nail. The constant current control may include an adaptable voltage constant current source, which may feature a constant current control loop which includes a sense input means, indicative of nail resistance which facilitates conversion of output voltage to the minimum required level that can generate the desired current through the nail, thereby minimizing energy drain from the power source. The device may be an iontophoretic device. The device may include at least one active substance, such as an antifungal agent and excipients, as suitable to facilitate iontophoretic delivery of the antifungal agent into and through the nail plate. A further aspect is a kit comprising an electrically operated device including a constant current source and a composition, comprising an antifungal agent. An additional aspect is a method of constant current control, which takes into consideration individual nail resistance, according to the current through the nail. Still, another aspect is the use of the electrically operated device with constant current control, in combination with the composition to treat onychomycosis.

In accordance with an embodiment of the invention, a device promotes delivery of a fixed dose of a compound from a composition using constant current to and/or through the nail plate and to the nail bed.

Before explaining at least one embodiment of the invention in detail, it is to be understood that the invention is not limited in this application to the details of construction and the arrangement of the components set forth in the following description or illustrated in the drawings. The invention is applicable to other embodiments of being practiced or carried out in various ways. Also, it is to be understood that the phraseology and terminology employed herein is for the purpose of description and should not be regarded as limiting.

The principles and operation of a device according to the present invention may be better understood with reference to the figure. The figure shows an exemplary embodiment of the present invention and is not limiting.

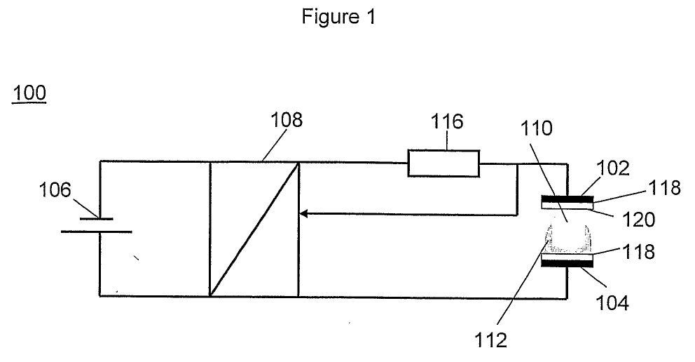

FIG. 1 shows a schematic view of an iontophoretic device including a constant current control means according to an embodiment of the present invention.

As can be seen from FIG. 1, device 100 includes at least one treatment device electrode 102, at least one counter electrode 104 and at least one power source 106 in spaced relation to each other.

In some embodiments at least one power source 106 is the power source for providing power to the device. In some embodiments power source is a battery. Power source 106 may be configured to facilitate an adaptable voltage and constant current source. In one embodiment, an adaptable voltage constant current source includes a controllable (variable) voltage converter 108, such as a controllable voltage DC/DC converter, which may be coupled to the power source 106. As used herein the term ‘coupled to’ includes directly coupled, indirectly coupled, electrically coupled, conductively coupled, ionically coupled, connected to and a combination thereof. In one non-limiting example two 1.5 V batteries may be used with a DC/DC converter which may provide any suitable voltage by stepping up or stepping down the battery voltage. Power source 106 may be coupled to the at least one active electrode 102, wherein the active electrode 102 may be disposed on the infected nail area 110 and the power source may also be electrically coupled to the counter electrode 104, wherein the counter electrode 104 may be disposed on a non-infected area of the body, such as on a non-infected part of the digit 112.

The resistance of the nail 110 may differ according to many factors, which may include but are not limited to age, sex, areas of infection, toenail or fingernail, thickness of nail, smoking, previous or current finger sucker, diet, physical trauma, body hydration etc. The nail 110 may contain areas of different resistance.

In the device of the present invention 100, a sensing means 116 or other suitable equivalent means 116 is electrically coupled in series to the nail 110. The sensing means 116 may be a resistor or any other suitable device for sampling current. The resistor may be any suitable resistor and may be made by any suitable method, such as for example using a printing technology. The sensing means 116 may sample the current and may adjust the voltage applied to the current source with high efficiency according to the nail resistance.

In some embodiments, the sensing means 116 may be coupled in a closed control loop to the adaptable voltage constant current source, which may include a controllable voltage converter 108 such as a controllable DC/DC converter 108 wherein the output voltage of the DC/DC converter is controlled in a closed loop by the current through the nail 110 that is sensed by the sensing means 116. In this closed system the current is maintained constant by taking into account from the sensed current the varying nail resistance and changing the voltage output accordingly in order to maintain a constant current through the nail. In an embodiment, the sensing means 116 may sample a current and provide the measurement of the sampled current to the voltage converter 108, which may then determine how much the current has deviated from a predetermined constant level and how much adjustment to the voltage is needed to maintain and/or return to a constant current and adjust the voltage source accordingly. Alternatively, the sensing means 116 may sample the current and determine how much the current has deviated from a constant level and then provide the deviation to the voltage converter 108, which may then determine how much adjustment to the voltage is needed and adjust the voltage source accordingly. Alternatively, a processor may be used in the device to perform the determinations for the sensing means, the voltage converter, or both.

This method facilitates a controllable voltage constant current source which is highly efficient and thereby minimizes the energy drain from the power source 106. In an alternative embodiment, the sensing means, such as a resistor 116 may be printed or integrally formed with one of the electrodes 102 and 104.

The current may be measured directly by the sensing means 116 sampling the current flowing therethrough. Alternatively, the current may be measured indirectly by the sensing means 116 sampling a related parameter, e.g., the amount of drug being administered to the nail, and then calculating the current from the sampled related parameter. Alternatively, the current may be measured both directly and indirectly by the sensing means 116 sampling the current and a related parameter and then calculating an average, for example, of the sampled results.

The device 100 may further comprise a conductive interface medium 118, which readily facilitates providing a conductive interfacing layer between the nail and the device 100 and can be configured as a conductive adhesive facilitating attachment of the device 100 to the skin/nail. The conductive interfacing layer may be disposed on at least one of the active electrode 102, the counter electrode 104 and the nail 110. Alternatively, the ionic conductive material may be accommodated in a holding device (not shown in figure), such as a retainer, which may be coupled to the device. One non-limiting example of a suitable conductive interface medium is a hydrogel.

The device 100 of the present invention may be used to deliver any suitable active substance/drug 120 into/onto/through the nail/skin. The term ‘active substance’ as used herein includes, but is not limited to any ‘active formulation’, ‘active composition’, ‘active agent’, pharmaceutical, drug, cosmeceutical, cosmetic substance, decorative substance, therapeutic substance, natural and synthetic; which has an effect on any nail and/or skin condition, such as, but not limited to a physical, physiological, medical, cosmetic, biochemical, biological, chemical condition or a combination thereof. The term ‘effect’ includes a therapeutic effect, a cosmetic effect, an inhibitory effect, a stimulatory effect, a physical effect, a biological effect, a physiological effect, a preventative effect, a placebo effect or a combination thereof.

In an embodiment, wherein device 100 is configured to deliver an active substance 120, such as an antifungal formulation to the nail/skin area, such as to the area of infection, the active substance 120 can be disposed in/on at least one of on the conductive interface medium 118, integrally part of the conductive interface medium formulation 118, on or integrally part of the active electrode layer 102 and on the nail/skin region 110.

Constant current of device 100 promotes delivery of a fixed dose of the active substance.

Device 100 may further include at least one substrate base layer (not shown in figure). In one embodiment, the at least one active electrode 102, the at least one counter electrode 104, the at least one power source 106, the controllable voltage converter 108, and the sense input means 116 are disposed on a substrate base layer in spaced relation to each other. In such an embodiment, device 100 may be configured thin and flexible and may be readily conformable to the contours of the digit and nail. In one embodiment, the device 100 is part of a kit and the parts of the kit may be assembled before use. Components of the kit may be disposable or reusable. In one embodiment of a kit the active formulation 120 and/or conductive interface medium 118 may be stored separately and integrally attached to device 100 before use.

In a further embodiment of the present invention, resistance data of the nail resulting from the sensing means 116 may be sampled and displayed and used to indicate whether resistance lowering treatment is needed. Non-limiting examples of resistance lowering treatment include, but are not limited to at least one of treatment with urea, poration of the nail, stripping of the nail, steam and treatment with acid.

It is to be understood that the iontophoretic device of FIG. 1 may include other electrical components, such as a capacitor, diode, transistor, series resistors, etc., and/or other configurations capable of providing a constant current according to an embodiment of the present invention.

In one embodiment, the present invention provides a method of delivering a constant current to at least one nail. The method includes the steps of applying a device as described in FIG. 1 such that the at least one active electrode is disposed on at least one affected nail. The device may be applied for any suitable length of time, which may in some embodiments be for extended periods of time. The sensing means may sample the current through the nail and facilitates adjustment of the power supply output voltage according to the sampled current, such that a constant current is supplied to the nail with high efficiency. In an embodiment of a method of treating a nail condition the method may further include promoting delivery by for example iontophoresis of at least one active substance to the at least one nail to treat the nail condition. The constant current may facilitate delivery of a fixed amount of the at least one active substance.

The device and method of the present invention can be employed in treatment of other areas of the body such as, but not limited to the skin, wherein constant current is used and wherein the body area characteristically displays variable resistance, in order to reduce wasteful power drain and facilitate long life of the battery.

One skilled in the art can appreciate from the foregoing description that the broad techniques of the embodiments of the present invention can be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

1. A device for treatment of a nail condition comprising:
at least one active electrode disposed on a nail;
at least one counter electrode;
at least one power source for providing power to the device, wherein the at least one power source is electrically coupled to the at least one active electrode and the at least one counter electrode;
a controllable voltage converter coupled to the at least one power source; and
a sensing device coupled to the nail and to the controllable voltage converter, wherein the sensing device is configured to facilitate adjustment of the output voltage of the power source according to the current through the nail with high efficiency.

2. The device of claim 1, wherein the nail condition is at least one of onychomycosis, a bacterial infection, viral infection and combination thereof.

3. The device of claim 1, wherein the nail is at least one of a toe nail, a finger nail, surrounding area of nail and a combination thereof.

4. The device of claim 1, wherein the device comprises an iontophoresis device.

5. The device of claim 1, wherein the controllable voltage converter is a DC to DC converter.

6. The device of claim 1, further comprising an active agent for treating the nail condition.

7. The device of claim 6, wherein the device promotes delivery of the active agent to the area of the nail condition.

8. The device of claim 6, wherein the constant current promotes delivery of a fixed dose of the active agent.

9. The device of claim 1 further comprising a conductive interface medium for facilitating a conductive interface between the device and the nail.

10. (canceled)

11. The device of claim 1, further comprising at least one substrate base layer.

12. The device of claim 1, wherein the sensing device comprises a resistor.

13. The device of claim 1, wherein the sensing device is coupled to the controllable voltage converter and to the nail in a closed loop.

14. The device of claim 1, wherein the sensing device samples the current through the nail.

15. The device of claim 1 as part of a kit.

16. A method of facilitating delivery of a constant current to a nail, comprising:
applying a device to a body area, the device comprising:
at least one active electrode disposed on the nail;
at least one counter electrode;
at least one power source for providing power to the device, wherein the at least one power source is electrically coupled to the at least one active electrode and the at least one counter electrode;
a controllable voltage converter coupled to the at least one power source; and
a sensing device coupled to the nail and to the controllable voltage converter, wherein the sensing device is configured to facilitate adjustment of the output voltage of the power source according to the current through the nail with high efficiency;
sampling current to the nail; and
based on the sampled current, adjusting power supply output voltage to facilitate supplying a constant current to the nail.

17. A method of treating a nail condition comprising:
applying at least one active substance to a nail body area with a device, the device comprising:
at least one active electrode disposed on the nail;
at least one counter electrode;
at least one power source for providing power to the device, wherein the at least one power source is electrically coupled to the at least one active electrode and the at least one counter electrode;
a controllable voltage converter coupled to the at least one power source; and
a sensing device coupled to the nail and to the controllable voltage converter, wherein the sensing device is configured to facilitate adjustment of the output voltage of the power source according to the current through the nail with high efficiency;
sampling current by the sensing device;
adjusting power supply output voltage according to the sampled current to facilitate supplying a constant current to the nail; and
promoting delivery of at least one active substance to the nail.

18. The method of claim 17, wherein the constant current facilitates delivery of a fixed amount of at least one active substance.

19. (canceled)

20. A device comprising:
a first electrode;
a second electrode;
a sensing device to sample current through the first and second electrodes;
a controllable voltage source; and
a controllable voltage converter in electrical communication with the sensing device to control the current at a constant level based on the sample from the sensing device.

21. The device of claim 20, wherein the controllable voltage converter controls the current at a constant level by adjusting the voltage output from the controllable voltage source.
